(19) United States
(12) Patent Application Publication
(10) Pub. No.: US 2026/0038690 A1
(43) Pub. Date: Feb. 5, 2026

(54) NEW SURVIVAL PROGNOSTIC INDEX IN GLIOBLASTOMA MULTIFORME BASED UPON ALTERATIONS OF THE CONNECTIONS OF THE BRAIN WHITE MATTER MEASURED WITH NMR DIFFUSION TECHNIQUES

(71) Applicant: Università degli Studi di Padova, Padova PD (IT)

(72) Inventors: Maurizio Corbetta, Padova (IT); Alessandro Salvalaggio, Padova (IT); Lorenzo Pini, Pianezze (Vicenza) (IT)

(73) Assignee: Università degli Studi di Padova, Padova PD (IT)

(21) Appl. No.: 18/996,227

(22) PCT Filed: Jul. 19, 2023
(86) PCT No.: PCT/IB2023/057357

§ 371 Date: Jan. 17, 2025

(30) Foreign Application Priority Data
Jul. 21, 2022 (IT) 102022000015360

(51) Int. Cl. G16H 50/30; G16H 30/20; G16H 50/20

(52) U.S. Cl. CPC G16H 50/30; G16H 30/20; G16H 50/20

(57) ABSTRACT

The present invention relates to a computer implemented method for determining a new prognostic index of a brain tumour, in particular glioblastoma. The new proposed index in particular is based upon the determination of alterations of the connections of the brain white matter measured with magnetic resonance diffusion techniques.

### FIELD OF THE INVENTION

The present invention relates to a computer implemented method for determining a new prognostic index for a brain tumour in a subject, in particular for glioblastoma. The new proposed index in particular is based upon the determination of alterations of the connections of the brain white matter measured with magnetic resonance diffusion techniques.

### STATE OF ART

Glioblastoma (GBM) is the most common malignant tumour of the brain, with global impact of about 3-4 cases out of 100,000 people per year. The prognosis of the patients affected by glioblastoma is unfavourable, with an average survival (overall survival, OS) lower than 15 months. To date the prognostic factors of brain tumours, such as glioblastoma, most used in clinical field and in the pharmacological research include factors linked to the patient (age, healthy state), to the tumour (histological and molecular profile) and to the treatment (extension of surgical resection, radiant therapies and performed chemotherapies).

However recent findings in the field of neurosciences suggest that different brain areas subtend peculiar properties in terms of connections with other areas. A region with high density of fibers of white matter, in particular, has a higher connection with other brain regions and lesions to this type of region can severely impact mental and cognitive status. Generally, the brain tumours are examined independently from the development brain seat, without considering the level of disconnection induced thereby. Among the prognostic factors of the brain tumours used to date, however, there is no biomarker taking into consideration the spatial type of the tumour in relation to the structural organization of the brain.

Besides in the field of the neurodegenerative diseases, diagnostic and prognostic tests for new drugs are currently under development, capable of changing the course of disease (for example, PET for amyloid in Alzheimer disease). Among the new developed methods of neuroimaging there are the development and the processing of the so-called big data. To date, public datasets result to be available, which make available thousands of brain images for the development of new atlases and methods. These normative atlases were applied to the study of the brain ictus, for example to estimate the impact of a specific lesion on the underlying brain connections. In the specific case, the mask of the segmented lesion in the patient is used to estimate a series of amounts which can be derived from these normative atlases. This approach is at the basis of the “indirect” estimate of disconnection induced by a lesion on the remaining brain, where disconnection means the number and map of fibers altered by the lesion. The disconnections are defined “indirect” since they are processed indirectly on a normative atlas which can represent a good proxy of the damage not directly calculable in the patient.

Although the neuroimaging piece of data currently results to be absolutely necessary for the diagnosis and surgical planning of the brain tumours, the new methods developed within the computational neurosciences have not yet been translated in applications of prognostic type of brain tumours as occurred, on the contrary, for the other above-mentioned neurological pathologies. One of the main reasons relates to the impossibility of estimating the level of connectivity impairment directly from the neuroimaging piece of data of the patient. In fact, the brain lesion involves a signal loss which does not allow to reconstruct accurately the damaged beams inside the tumour mass. In a study of 2015, Mickevicius et al. have examined the relationship existing between the seat of a GBM which results to intersect specific tracts of white matter at brain level and the prognosis of GBM (“Location of brain tumour intersecting white matter tracts predicts patient prognosis” 2015, J. of Neuro-Oncology, vol. 125, Nr. 2). The prognostic evaluation is performed in such study by comparing the average survival (overall survival) of a group of patients whose GBM results to intersect white matter tracts of specific brain areas (that is, in the region of the corpus callosum or bilateral corticospinal bundle, right inferior front-occipital bundle, right anterior thalamic radiation) with a group of patients whose GBM does not intersect such specific brain tracts. The method of Mickevicius et al. then provides an “ad hoc” or “a priori” selection of the white matter tracts intersecting the brain tumour considered relevant for prognostic purposes, starting from the assumption that the brain position in the above-mentioned brain tracts is on itself related with the average survival of the patient. However, the above-mentioned method does not provide to determine a prognostic index calculable in automated way and capable of allowing a prediction of the average survival also in patients in which the brain tumour does not intersect the particular selected tracts.

Therefore, even by considering the fact that the disconnection damage following a tumour is different depending upon the brain seat, the development of new indexes and methods for a more accurate prognosis of such lesions or pathologies appear to be urgent.

### SUMMARY OF THE INVENTION

The aim underlying the present invention is to provide a computer implemented method for determining a prognostic index for a brain tumour, in particular for glioblastoma, allowing to obviate the problems found in the field, with particular reference to the poor consideration of the disconnection level of the nervous fibres induced by such lesions or pathologies.

The new methods developed by the authors of the invention allows to determine, starting from an image of a brain lesion or pathology, a prognostic index of structural density which represents an indirect estimation of the disconnections of nerve fibers, in particular fibers of brain white matter, induced by the considered particular lesion or pathology.

The construction of such index is based on the determination of the total value, with respect to a normative atlas, of the number of the tracts of nerve fibers passing through each voxel of the analysed brain image, belonging to the area of the brain affected by said lesion or pathology.

As it will be illustrated in the subsequent detailed description, the new structural density index can be calculated by using, as reference normative atlas, diffusion images obtained by a dataset of healthy controls, starting therefrom it is possible to generate a normative density map of the white matter consisting of the average number of tracts of nervous fibers passing through each voxel. Advantageously, by exploiting the information related to the density of nerve fibers at brain level derived from the reference normative atlas, the method, the invention relates to, allows to estimate indirectly the damage caused by a lesion or pathology in a patient at the level of brain connection.

According to the method of the invention, when a brain tumour results to be localized in brain regions with high density of fibres, calculated starting from the sum of the average number of tracts of nervous fibers passing through the brain area involved by the tumour, the prognosis is worse. In other terms, the method, the present invention relates to, allows to obtain one single number, called as Tract Density Index (TDI), calculable in automated way as prognostic index and capable of allowing to predict the average survival of patients affected by brain tumours.

Not only the authors of the invention have found a correlation between the so-calculated TDI index and the average survival of the patients affected by brain tumours, but they have also verified that the determination of the TDI index allows to increase the prediction accuracy with respect to the commonly used known prognostic factors (MGMT, age, performance status, surgery extension).

As it will be clearly highlighted in the experimental section of the present application, the results obtained by the authors of the invention have surprisingly demonstrated that the new structural density prognostic index determined with the method of the present invention allows to predict accurately the average survival of patients affected by brain tumours, in particular by glioblastoma. The method, the invention relates to, in fact has shown a high accuracy level (that is 87%) calculated in an independent cohort.

The relation existing between the specific structural density index and the average survival of patients affected by brain tumours under preliminary study, observed for the first time by the authors of the invention, demonstrates that different tumours can impact and damage brain connections peculiarly, with impacts on patient prognosis.

Advantageously, what found by the authors of the present invention about the relationship existing between the specific TDI structural density index and the average survival, allows to extend the processing of such prognostic index to all patients affected by brain tumour and not only to the patients in which the tumour results to intersect the nerve fibre tracts in specific brain areas, as described instead in the above-discussed work of Mickevicius et al.

To the purpose of calculating the prognostic index, the method according to the present invention further allows to consider the whole tumour area and not, as provided in Mickevicius et al., only the tumour portion which localizes in a determined brain tract. This makes that the method, the present invention relates to, can be automated and does not require expert supervision, required instead for executing the method of Mickevicius et al. with the purpose of “ad hoc” selecting the white matter bundles most suitable/relevant for prognostic purposes.

The method, the present invention relates to, then can be implemented in a software, preferably with user-friendly graphic interface (GUI), through which different users can easily analyse a segmented and normalized mask of a brain tumour of a patient, so as to obtain the structural density prognostic index and to predict the average survival of the patient more accurately, even by comparison with reference indexes. The prognostic index which can be determined by the methods, the invention relates to, can also be used as biomarker within the evaluation of new therapies of the disease-modifier type (at level of clinical trials), in addition to the generally considered “classical” prognostic variables.

The invention then relates to a computer implemented method for determining a prognostic index for a brain tumour in a subject, comprising the following steps:
a) receiving as input a three-dimensional nuclear magnetic resonance image of the brain of said subject, in which said three-dimensional image 1 is segmented and recorded in a reference space and comprises a number n of voxels 10, each one having an intensity value indicative of the belonging or extraneity of the aforementioned voxel to an area of the brain bearing said brain tumour; in which said three-dimensional image 1 is a binary image in which each voxel 10 has an intensity value equal to 1 when belonging to said area of brain affected by the brain tumour and has an intensity value equal to 0 when extraneous to the aforesaid area;
b) receiving as input a three-dimensional reference cerebral nuclear magnetic resonance image 2, in which said reference image 2 is recorded in said reference space and comprises a number m of voxels 20, in which m=n, each one bearing a value indicative of the number of tracts of nerve fibers passing through it;
in which said reference image 2 corresponds to an average of digital tractography images obtained from a plurality of healthy subjects by diffusion-weighed magnetic resonance, and in which each voxel 20 of said reference image 2 bears a value indicative of the average number of tracts of nerve fibers passing through it
c) determining the three-dimensional coordinates of each voxel 10 of the image 1 of the brain of said subject having an intensity value indicative of the belonging of the aforementioned voxel to an area of the brain bearing said brain tumour;
d) extracting, for each voxel 20 of the reference image 2 having the three-dimensional coordinates determined in step (c), said value indicative of the number of tracts of nerve fibers passing through it;
e) adding the values extracted in step d) so as to obtain said prognostic index and comparing said prognostic index with a cut-off prognostic index determined for a group of subjects whose average survival is known,
in which, when said prognostic index has a higher value than said cut-off prognostic index, said subject is classified as having a low average survival, or, when said prognostic index has a lower value than said cut-off prognostic index, said subject is classified as having a high average survival.

The invention also relates to:
a computer programme comprising a list of instructions which, when executed on a computer, implement the steps of a method according to any one of the herein described embodiments;
a device for implementing the method according to any one of the herein described embodiments, in which a computer programme according to any one of the herein described embodiments is stored.
Other advantages and features of the present invention will result evident from the following detailed description.

### BRIEF DESCRIPTION OF FIGURES

FIG. 1. Image representing schematically a method for determining a prognostic index for a brain tumour in a subject according to a first embodiment of the present invention.

FIG. 2. Image representing schematically a method for determining a prognostic index for a brain tumour in a subject according to a second embodiment of the present invention.

FIG. 3. Image representing schematically a method for determining a prognostic index for a brain tumour in a subject according to a third embodiment of the present invention.

### GLOSSARY

The terms used in the present description are as generally understood by the person skilled in the art, unless otherwise indicated.

In any point of the present description and claims, under the expression “receiving as input a three-dimensional nuclear magnetic resonance image of the brain of said subject” one means receiving as input a first set of data related to a three-dimensional nuclear magnetic resonance image 1 of the brain of said subject, segmented and recorded in a reference space, in which said first set of data comprises a plurality of the intensity values corresponding to a number n of voxels 10 of said image, each intensity value being indicative of the belonging or extraneity of a determined voxel 10 of the image to an area of the brain bearing said brain tumour.

In any point of the present description and claims, under the expression “receiving as input a three-dimensional reference cerebral nuclear magnetic resonance image 2” one means receiving as input a second set of data related to a reference cerebral nuclear magnetic resonance image 2, recorded in said reference space, in which said second set of data comprises a plurality of intensity values corresponding to a number m of voxels 20 of said reference image, in which m=n, each intensity value being indicative of the number of tracts of nervous fibers passing through each voxel 20.

In the context of the present invention, under the term “voxel” the measurement unit of the volume of a three-dimensional digital image is meant, and it represents the three-dimensional counterpart of the bidimensional pixel.

Under the expression “average survival” or “overall survival” (also known as overall survival OS), in the context of the present invention, the time period elapsing between the date of the first surgery thereto the subject affected by brain tumour is subjected and the moment of death of the subject is meant.

The expression “brain tumour” in the context of the invention relates to a tumour of the central nervous system, then the set of brain, medulla oblongata and cerebellum, generally represented by a mass consisting of cells of various origin which develop in the brain of a subject and which, due to their presence, alter or can compromise the function of different brain areas.

In the context of the present description, under the term “subject” preferably a mammal, still more preferably a human being is meant.

In the present description of the invention and in claims, when referred to a three-dimensional nuclear magnetic resonance image, segmented and recorded in a reference space according to any one of the herein described embodiments, the term “image” can also be replaced by the term “mask”.

In the present description of the invention and claims, when referred to a reference cerebral nuclear magnetic resonance image, recorded in a reference space, according to any one of the herein described embodiments, the term “reference image” can also be replaced by the term “template” or “reference template”.

The area of impregnation of contrast medium and the possible “central necrosis of the brain tumour is also defined in the present description as “tumour core”.

In any point of the present description and claims, the prognostic index which can be determined through a method according to any one of the herein described embodiments can be designated as “Tracts Density Index” or “TDI”.

In any point of the present description and claims, the term “comprising” can be replaced by “consisting of”.

### DETAILED DESCRIPTION OF THE INVENTION

A first aspect of the present invention relates to a computer implemented method for determining a prognostic index for a brain tumour in a subject, comprising the following steps:
a) receiving as input a three-dimensional magnetic resonance image 1 of the brain of said subject, in which said three-dimensional image 1 is segmented and recorded in a reference space and comprises a number n of voxels 10, each one having an intensity value indicative of the belonging or extraneity of the aforementioned voxel to an area of the brain bearing said brain tumour;
b) receiving as input a three-dimensional reference cerebral magnetic resonance image 2, in which said reference image 2 is recorded in said reference space and comprises a number m of voxels 20, in which m=n, each one bearing a value indicative of the number of tracts of nerve fibers passing through it;
c) determining the three-dimensional coordinates of each voxel 10 of the image 1 of the brain of said subject having an intensity value indicative of the belonging of the aforementioned voxel to an area of the brain bearing said brain tumour;
d) extracting, for each voxel 20 of the reference image 2 having the three-dimensional coordinates determined in step c, said value indicative of the number of tracts of nervous fibers passing through the above-mentioned voxel 20;
e) determining said prognostic index on the basis of the values extracted in step (d).

As previously mentioned, the authors of the invention have found that an index which can be determined according to any one of the methods illustrated in the present description and claims is useful in the formulation of an accurate prognosis for subjects affected by brain tumour. In particular, the prognostic index which can be determined by any method of the present invention is useful to determine or predict the average survival also known as “overall survival” of the subject affected by brain tumour, also designated in the present application under the expression inglese overall survival (OS).

According to an aspect of the invention, said brain tumour is a primitive tumour of the central nervous system which develops directly in the central nervous tissue or a secondary tissue, or metastasis, which originates from tumours growing in other organs (for example, lung or breast) and which then spread to the nervous tissue.

The primitive tumours of the central nervous system include a wide set of pathological entities. Not limiting examples of primitive brain tumours include tumours originating from the glial cells, that is glial tumours or gliomas, or not glial tumours. The glial tumours are classified in different sub-types, which can be distinguished based upon the cell type therefrom they originate and the differentiation or malignity level.

According to an aspect of the present invention, said brain tumour is a glial tumour selected from astrocytomas (originating from astrocytic cells), oligodendrogliomas (originating from oligodendroglial cells) and ependymomas (originating from ependymal cells), preferably said brain tumour is selected from the group consisting of: diffuse astrocytoma, gemistocytic astrocytoma, anaplastic astrocytoma, glioblastoma, giant cell glioblastoma, gliosarcoma, epithelioid glioblastoma, pleomorphic xanthoastrocytoma, oligodendroglioma, diffuse oligodendroglioma, anaplastic oligodendroglioma, oligoastrocytoma and anaplastic oligoastrocytoma.

According to an additional aspect of the invention, said brain tumour is a non-glial tumour selected from medulloblastoma, meningioma, primitive lymphoma of the central nervous system, pituitary tumour.

According to a preferred aspect of the invention, said brain tumour is the glioblastoma also known and designated in the present application with the abbreviation GBM or as grade IV glioblastoma multiforme or astrocytoma, less commonly known also as polymorphous glioblastoma.

Glioblastoma is a very aggressive form of tumour affecting the central nervous system and which appears often with headaches of increasing intensity, nausea, vomiting and seizures. Such disorders are caused by the tumour mass which, by expanding inside the skull, causes a pressure increase and the dilatation of the cerebral blood vessels. The disorders (symptoms) associated thereto can also be of neurological type and not specific, for example disorders of personality and state of consciousness.

Consisting of an heterogenous set of poorly differentiated astrocytic tumour cells, glioblastoma mostly affects adults and usually appears in the cerebral hemispheres; less frequently in the brainstem or spinal cord. As all brain tumours, except very rare cases, it does not expand beyond the structures of the central nervous system. Glioblastoma can develop from a diffuse astrocytoma (grade II) or from anaplastic astrocytoma (grade III) (in such case it is called secondary), but more frequently it appears de novo, without any evidence of previous neoplasia (in this case it is called primary).

Under the macroscopic profile, glioblastoma can occupy more than a cerebral lobe. Generally, the lesion is unilateral, but those of the brainstem and the corpus callosum can be bilaterally symmetrical. In this case, the tumour occupies the same position in the two hemispheres and appears with a “butterfly”-like aspect. The bilateral supratentorial extension is due to a quick growth along the myelinated structures, in particular through the corpus callosum and along the fornices towards the temporal lobes. The boundaries of the neoplastic mass, generally, are instead blurred everywhere. The colour is greyish, but abundant variegations can be found, caused by necrosis or more or less recent haemorrhages, therefore yellowish areas appear on the grey background, due to fatty degeneration or necrosis and reddish or blackish areas due to haemorrhages.

The peripheral area of hypercellular tumour tissue appears as a soft and grey rima. The necrotic tissue can also border adjacent brain structures without a macroscopically detectable tumour intermediate area. Glioblastomas can be also speckles with red and brown spots due to haemorrhages, which sometimes are quite wide to cause symptoms similar to the stroke, which can constitute the first clinical sign of tumour. Macroscopic cysts, when present, contain a cloudy fluid coming from liquefied necrotic tumour tissue, clearly in contrast with the well-delineated retention cysts of grade II diffuse astrocytomas. Most glioblastomas of the cerebral hemispheres are clearly intraparenchymal, with epicentre in the white matter. Sometimes the neoplasia appears like widely superficial and in contact with leptomeninges and dura mater and can be mistaken for metastatic cancer or an extra-axial lesion such as meningioma.

Generally, the brain area involved by central necrosis of glioblastoma can occupy more than 80% of the total mass of the brain tumour.

As mentioned, step a) and step b) of the method, the present invention relates to, require respectively receiving, as first and second input piece of data, at least a nuclear magnetic resonance image 1 of the brain of the subject affected by said brain tumour, which image 1 is segmented and recorded in a reference space, and at least a reference cerebral nuclear magnetic resonance image (2) recorded in the same reference space, that is a space of three-dimensional coordinates.

The image 1 of the subject brain and the reference image 2 then are, both, three-dimensional images, respectively characterized by a number n of voxels 10 and a number m of voxels 20, where n=m, each one identifiable through a set of special coordinates x, y and z.

In other terms, each voxel of the considered three-dimensional image can be identified through a set of coordinates x,y,z, expressing the position of the voxel in the three-dimensional space of the image.

Each voxel of the three-dimensional image 1 and image 2 has a numeral value associated thereto, which represents an independent measurable or variable property related to the object lying in the volume unit of the three-dimensional image represented by the particular voxel having a given set of three-dimensional coordinates.

In particular, as above mentioned and as will be also illustrated in details hereinafter, each voxel 10 of the image 1 has an intensity value indicative of the belonging or extraneity of the aforementioned voxel to an area of the brain bearing said brain tumour; each voxel 20 of the reference image 2 instead has a value indicative of the number of tracts of nerve fibers passing through it.

The image 1 can be obtained starting from the acquisition of a nuclear magnetic resonance image 1′ of the subject affected by brain tumour by using any one of the magnetic resonance imaging techniques known to an expert in the field.

The nuclear magnetic resonance, also known and designated in the present application under the abbreviation NMR, provides three-dimensional images of the brain of the subject subjected to the analysis, which allow to detect the seat, the sizes, the disease extension and the ratios with the surrounding structures. The use of advanced methods in magnetic resonance, such as the analysis of diffusion and perfusion, as it also will be illustrated hereinafter, can provide additional information about cellularity and vascularization of the analysed cerebral regions.

According to an aspect of the invention, the image 1 can be obtained starting from the acquisition of a nuclear magnetic resonance image 1′ of the brain of said subject by using a contrast agent or means, in particular gadolinium.

In particular, the image 1 can be obtained starting from the acquisition of a nuclear magnetic resonance image 1′ of the brain of said subject, by applying any sequence of magnetic resonance used within the magnetic resonance imaging, where under “magnetic resonance sequence” a particular configuration is meant, which can be obtained through apparatus for magnetic resonance, which comprises a specific series of pulses and/or field gradients.

Preferably, said image 1 according to any one of the herein described variants is obtained starting from a magnetic resonance image 1′ of the brain of said subject weighed in T1 or weighed in T2, with or without application of a contrast agent, where T1 and T2, respectively, relate to the longitudinal relaxation (or spin lattice, that is recovery of the magnetization in the same direction of the static magnetic field) and cross relaxation (that is spin-spin, transversal to the static magnetic field).

As an expert in the field knows, in order to create a T1-weighed image, the magnetization has to recover before measuring the signal by changing the repetition time (TR). This image weighting is useful to evaluate the brain cortex and, generally, to obtain morphological information, as well as for post-contrast imaging. In order to create a T2-weighed image, the magnetization can decay before measuring the MR signal by changing the eco time (ET). This image weighting is useful to detect oedema and inflammation, by revealing lesions of white matter and by evaluating the anatomy of organs such as prostate and uterus.

According to an aspect of the invention, the execution of magnetic resonance in said subject affected by brain tumour, aimed at generating said image 1′, comprises at least one T1-weighed axial or coronal sequence without gadolinium, T1-weighed multiple sequences with gadolinium according to the three axes, and T2-weighed sequences and/or sequences of (generally axial or coronal) Fluid Attenuated Inversion Recovery (FLAIR) type. As a person skilled in the art knows, FLAIR sequences are sequences of pulses with inversion recovery used to annul the signal coming from the fluids. For example, within the brain imaging, this sequence can be used to suppress the signal of the cerebrospinal fluid (CSF).

As already mentioned, in order to obtain an image 1 according to any one of the herein described variants, the nuclear magnetic resonance image 1′ of the brain acquired for the subject affected by brain tumour subjected to analysis according to any one of the known techniques, as those exemplified in the present description, is subjected to a procedure of (i) segmentation and (ii) recording in a reference space.

The procedure of (i) segmentation generally consists of identifying, within the magnetic resonance image, the area or areas of the brain involved by the brain tumour with respect to the surrounding remaining brain tissue which is apparently unharmed, that is unrelated to the lesion, as well as, optionally, with respect to the areas involved by oedema or inflammation.

The segmentation procedure can be performed by using any one of the techniques known to a person skilled in the art, for example by means of a manual tracking procedure, that is operator-depending procedure.

The image 1 received as input according to any one of the herein described embodiments in particular is segmented so that each voxel 10 constituting said image 1 has an intensity value indicative of the belonging or extraneity of the aforementioned voxel to an area of the brain bearing said brain tumour.

Preferably, the image 1 received as input in step a) of a method according to any one of the herein described embodiments is segmented so that each voxel 10 constituting said image 1 has an intensity value indicative of the belonging or extraneity of the aforementioned voxel to the area of the brain involved by the central necrosis of the brain tumour, or tumour core.

According to an additional aspect of the invention, said image 1 received as input is segmented so that each voxel 10 constituting said image 1 has an intensity value indicative o of the belonging of the aforementioned voxel to one or more areas of the brain bearing necrosis, of the belonging of the aforementioned voxel to one or more areas of the brain bearing oedema, or of the extraneity of the aforementioned voxel to the areas at issue.

In a preferred embodiment of the method according to the invention, the image 1 provided as input in step a) of a method according to any one of the herein described embodiments is a binary image, in particular a T1-weighed image, in which each voxel 10 has an intensity value equal to 1 when belonging to said area of brain affected by the brain tumour and has an intensity value equal to 0 when extraneous to the aforesaid area.

Preferably, the image 1 provided as input in step a) of a method according to any one of the herein described embodiments is an image, in particular a T2-weighed image, in which each voxel 10 has an intensity value equal to 1 when belonging to one or more areas of the brain of said subject affected by necrosis, preferably central necrosis or tumour core, it has an intensity value equal to 2 when belonging to one or more areas of the brain of said subject affected by oedema, and it has an intensity value equal to 0 when extraneous to the aforesaid areas.

The procedure of (ii) recording or registration of a three-dimensional image according to any one of the herein described variants in a reference space, is a spatial normalization procedure providing that the above-mentioned three-dimensional image is projected or normalized in a standard space under analysis of magnetic resonance. Since a brain varies very much in size and shape depending upon the considered subject, an objective of the spatial normalization is to deform the images provided or processed starting from the brain scans so that an anatomical area shown by the brain scan of a considered subject corresponds to the localization corresponding in the scan of another subject. In other terms, at the end of the recording process in a same reference space, the co-recorded three-dimensional brain images are characterized by the same number of voxels, each one identifiable through the same set of spatial coordinates x, y, and z.

In the method, the present invention relates to, the image 1 and the reference image 2 according to any one of the variants illustrated in the present description and claims provided as input, are co-recorded in a common reference space. Due to the effect of such co-recording, each voxel belonging to a brain area in the image 1 can be identified through the same set of three-dimensional coordinates x, y and z of the voxel corresponding to the same brain area in the reference image 2.

According to an aspect of the present invention, said image 1 and said reference image 2 are recorded in the space of the Montreal Neurological Institute (MNI).

The procedure of recording or normalization of three-dimensional images according to any one of the herein described variants in a reference space can be performed according to any one of the techniques known to a person skilled in the art within the digital processing of brain images. By pure way of example, the spatial recording or normalization can provide the application of a three-dimensional not rigid transformation model, or “warp-field” for the “warping” procedure in the analysis of images of a brain scan with respect to a template or reference image. The warp-field can be parametrized by base functions such as cosine and polynomials.

According to an aspect of the invention, the image of reference brain nuclear magnetic resonance 2 provided as input in step b) of a method according to any one of the herein described variants is obtained starting from an atlas of tractographies obtained from a plurality of healthy subjects.

Tractography is a three-dimensional modelling technique used in neurosciences to represent visually the neural tracts, by using data collected by the imaging with diffusion tensor (DTI), special techniques of magnetic resonance and image information analyses. Such reference image or template then constitutes in this context the reference standard to identify the number of tracts of nervous fibers passing on the average by a specific voxel in a population of healthy subjects (that is normative population).

An atlas of tractographies suitable to be used for generating a reference brain image 2 according to any one of the herein described variants can be obtained according to any one of the known techniques to an expert in the field, for example starting from a data-set of publicly accessible tractographies available in a repository.

By pure way of example, said atlas can include tractography images obtained from at least 50 or at least 100 healthy subjects, preferably at least 180 healthy subjects.

According to an additional aspect of the invention, said reference brain image 2 according to any one of the herein described variants is obtained starting from a data-set comprising at least 5, at least 10, at least 20, at least 30, at least 50, at least 70, at least 80, at least 90, at least 100, at least 130, at least 150, at least 180 tractography images of healthy subjects.

As previously mentioned, each voxel 20 of said reference image 2 then bears a value indicative of the average number of tracts of nerve fibers passing through it. According to an additional aspect of the invention, said reference image 2 corresponds to an average of digital tractography images obtained from a plurality of healthy subjects by diffusion-weighed magnetic resonance, and each voxel 20 of said reference image 2 bears a value indicative of the average number of tracts of nerve fibers passing through it.

Preferably, in the context of the present invention, under the expression “tracts of nerve fibres” tracts of fibres of white matter are meant. In anatomy the white matter is given by the beams of (both ascending and descending) nerve fibres joining the brain and the spinal cord. The beams appear white due to the coating given by myelin.

Therefore, in a preferred embodiment, said reference image 2 corresponds to an average of digital tractography images obtained from a plurality of healthy subjects by diffusion-weighed magnetic resonance, and each voxel 20 of said reference image 2 bears a value indicative of the average number of tracts of fibres of white matter passing therethrough.

According to an aspect of the invention, the method according to any one of the herein described embodiments further comprises the following steps preceding said step a):
a′) receiving as input a magnetic resonance image 1′ of the brain of said subject and subjecting it to a segmentation process according to any one of the techniques known to a person skilled in the art such as those exemplified in the present description, so as to obtain a segmented three-dimensional image 1″ that is not normalized; and a″) recording said segmented image 1″ that is not normalized in said reference space according to any one of the herein described embodiments, in such a way as to obtain said image 1.

According to an additional aspect of the invention, a method according to any one of the herein described embodiments further comprises the following steps, preceding said step b):
b′) receiving as input a plurality of digital tractography images 2′ obtained from a plurality of healthy subjects and processing said images so as to obtain a reference image 2″ that is not normalized; and
b″) recording said reference image 2″ in said reference space, that is in the same reference space used in the procedure for recording or normalizing the brain image 1 according to any one of the herein described variants, in such a way as to obtain said reference image 2.

Preferably, in step b′) the processing of the tractography images provides determining an image corresponding to the average of the above-mentioned tractography images.

As also illustrated by pure way of example in the experimental section of the present application, the procedure for processing the prognostic index performed in step e) of a method according to any one of the herein described variants, is based upon the determination of the number of tracts of nervous fibers, in particular of the number of tracts of fibres of white matter, passing through each voxel 20 of the reference image 2 having three-dimensional coordinates corresponding to the coordinates of a voxel 10 of the analysed brain image 1 which results to belong to an area of the brain bearing said brain tumour. Such determination then provides the execution of steps c) and d) of the method according to the present invention.

The execution of step c) in particular allows to identify all voxels 10 of the brain image 1 according to any one of the herein described variants having an intensity value indicative of the belonging of the aforementioned voxel to an area of the brain bearing said brain tumour. Once identified the positions, and then the three-dimensional spatial coordinates of the above-mentioned voxel 10, the execution of step d) allows to extract, for each voxel 20 of the reference image 2 having the same three-dimensional coordinates of the voxels 10 identified in step c), the corresponding value indicative of the number of tracts of nerve fibers passing through it.

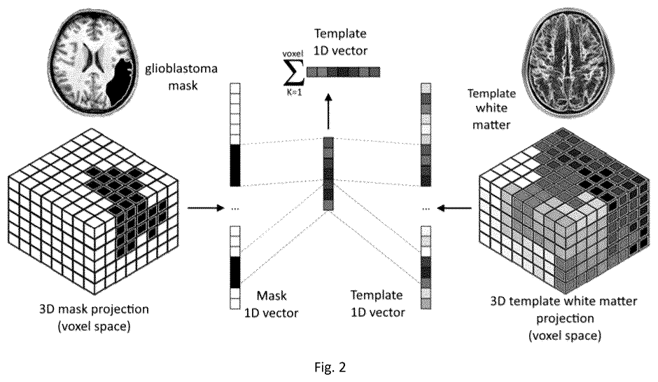

According to an aspect of the invention, step c) of a method according to any one of the herein described embodiments provides the execution of a step for the vectorization, respectively, of the brain image 1 and of the reference image 2 according to any one of the herein illustrated variants, that is the transformation of the above-mentioned three-dimensional images in a corresponding unidimensional vector. Such vectorization can be performed for example according to the schematized procedure by way of example in FIG. 2.

The vectorization step consists in a transformation of the spatial coordinates of each voxel of the brain image 1 and of the reference image 2. Such transformation does not alter the relationship between the voxels in the two images; therefore, the voxels which in the three-dimensional space of the image 1 of the brain bearing the brain tumour and of the reference image 2 or template are represented by the same set of spatial coordinates [x,y,z], assume the same value of coordinates [j] in the corresponding generated vector. Then, the vectorization step does not apply transformations to the value of each voxel of the processed images, but it transforms or projects the spatial coordinates thereof in a unidimensional space.

Preferably, in step c) of a method according to any one of the herein described embodiments, the three-dimensional image 1 according to any one of the herein illustrated variants is converted into a first vector v with length n=m, in which, for the index j varying from 1 to n=m, each element v, of the vector v bears an intensity value of a voxel 10 of the image 1 having a given set of three-dimensional coordinates; in the same step c), the reference image 2 according to any one of the herein described variants is converted into a second vector r, with length n=m, in which, for the index j which varies from 1 to n=m, each element r, of the vector r bears a value indicative of the number of tracts of nerve fibers passing through a corresponding voxel 20 of the reference image 2 having the same set of three-dimensional coordinates of the considered voxel 10.

According to an aspect of the invention, in step d) of a method according to any one of the herein described embodiments, the positions [j] in the vector r, generated starting from the reference image 2, are identified, corresponding to the positions [j] in the vector v, generated starting from the image 1, which correspond to intensity values indicative of a brain area bearing said brain tumour, for example values equal to “1”.

According to an aspect of the invention, still in step d), the values corresponding to these positions [j] are extracted by the vector F and stored in a third vector p with length n′ equal to the number of voxels 10 of said image 1 belonging to said area of the brain bearing said brain tumour.

Preferably, step e) of a method according to any one of the herein described embodiments, comprises adding the values extracted in step d) according to any one of the variants illustrated in the present description so as to obtain said prognostic index.

A preferred embodiment of a method according to any one of the herein described variants then comprises the following steps:
c′) converting said image 1 and said reference image 2 respectively into a first vector v̅ and in a second vector r̅, with length n=m, in which, for the index j which varies from 1 to n=m, each element v̅ⱼ of the vector v̅ bears an intensity value of a voxel 10 of the image 1 having a given set of three-dimensional coordinates, and each element r̅ⱼ of the vector r̅ bears a value indicative of the number of tracts of nerve fibers passing through a corresponding voxel 20 of the reference image 2 having the same set of three-dimensional coordinates of the considered voxel 10;
c″) identifying, in said vector r̅, each element r̅ⱼ that corresponds to an element v̅ⱼ having an intensity value indicative of the belonging to the voxel 10 of the image 1 to the area of the brain bearing said brain tumour;
d′) generating a third vector p̅ with length n′ equal to the number of voxels 10 of said image 1 belonging to said area of the brain bearing said brain tumour, in which each element p̅ᵢ corresponds to said element r̅ⱼ as identified in step c″);
e′) adding the values of each element p̅ᵢ in such a way as to obtain said prognostic index.

According to an aspect of the invention, a method according to any one of the herein described embodiments comprises an additional step of predicting and/or calculating the overall survival of said subject on the basis of said prognostic index.

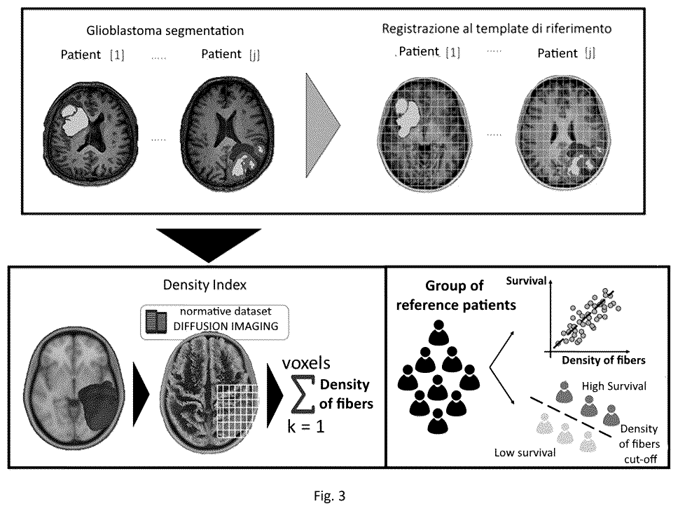

As schematically illustrated by pure way of example in FIG. 3, said prediction and/or calculation of the average survival of the subject affected by brain tumour can be performed by using any algorithm of artificial intelligence or of deep learning accessible to a person skilled in the art, by using as input the value of said prognostic index.

According to an aspect of the invention, a method according to any one of the herein described embodiments comprises a step in which said prognostic index is determined for a group of subjects whose average survival is known and used in a regression model to calculate the mathematic parameters of such model. Said parameters can be subsequently applied to new subjects in order to obtain a survival estimate at time of the magnetic resonance. The mathematic parameters of such model (or other more complex models) then can be updated as the sample of analysed subjects increases, with the purpose of making the model more and more accurate to predict the average survival.

According to an additional aspect of the invention, a method according to any one of the herein described embodiments comprises a step in which said prognostic index is determined for a sample or group of subjects thereof the average survival is known and parametrized according to a binary value (cut-off), as identified in said sample or group of subjects. Preferably, said prognostic parametrized index or cut-off corresponds to a determined percentile of the distribution in the sample of subjects subjected to analysis by means of the method of the invention.

According to an additional aspect, the method according to any one of the herein described embodiments comprises at least a step of comparing said prognostic index with a cut-off prognostic index determined for a sample or group of subjects thereof the average survival is known; in which said cut-off prognostic index corresponds preferably to a percentile of the distribution in said sample or group of subjects.

According to an aspect of the invention, when said prognostic index has a higher value than said cut-off value, the subject is classified as having a low survival level or low average survival, or when said prognostic index has a lower value than said cut-off value, said subject is classified as having a high survival level or high average survival.

In other terms, the parametrized prognostic index according to any one of the herein described modes can be applied to a new subject. A higher or lower value of said prognostic index (that is density index) in the new subject offers a predictive measure of the low or high survival level based on the sample of subjects and of the percentile taken as reference.

The present invention also relates to a computer programme comprising a list of instructions which, when executed on a computer, implement the steps of a method according to any one of the herein described embodiments.

The invention also relates to a device for implementing the method according to any one of the herein described embodiments, in which the computer programme according to any one of the herein described embodiments is stored.

### EXAMPLES

Some not limiting embodiment examples of the method according to the present invention are herewith reported by way of illustration.

### Example 1—Method for Determining a Prognostic Index Called Tracts Density Index (TDI) According to an Embodiment of the Present Invention

### Subjects, Considered Variables and Images Used in the Present Study

The study was performed according to a retrospective design on a clinical population. Two independent data-sets obtained from patients affected by glioblastoma were included for validating the prognostic index. A first data-set (defined as “discovery” dataset) consists in the data of magnetic resonance of patients belonging to the “Azienda Ospedale Università” in Padua (AOUPD) and to the “Istituto Oncologico Veneto” (IOV) in Padua. A second data-set (defined as “replicative” data-set) includes data of patients belonging to the Department of Neurosurgery—Charité Universitaetsmedizin, Berlin (Germany).

The patients of the discovery data-set were included based upon the following criteria:
i) siteological diagnosis of glioblastoma (glioma of grade IV according to classification WHO 2016);
ii) availability of pre-surgical images of magnetic resonance (with at least one of the following sequences: t2-weighed, Fluid Attenuated Inversion Recovery, T1-weighed both pre- and post-contrastographic images);
iii) the availability of clinical data of outcome (average survival or overall survival, OS).

Patients with:
i) magnetic resonance with low intensity of magnetic field (<than 1.5 T); ii) lack of acquisition of the axial plane, required to perform segmentation;
iii) presence of artefacts to the piece of data of resonance or with partial scan of brain;
iii) evidence of preceding diagnostic or therapeutic brain surgical procedures were excluded.

The replicative data-set included patients for which only t1-weighed (pre- and post-contrastographic) images were acquired. The other inclusion/exclusion criteria were analogous to those adopted for the dataset.

### Preparatory Procedures for Determining the Prognostic Index

The method for determining the prognostic index according to the present invention, as it will be illustrated hereinafter, requires the following input data:
i) images or masks of the brain tumour of a subject segmented in the native space and normalized in a reference space (this first step allows to transform the tumour of each patient into an image or mask in a normalized and common space); and
ii) a reference image or normative map of the number of fibres passing through each volume unit (voxel) of the map which has to be necessarily in the same reference space of the tumour images or masks (this second step allows to obtain a value for each voxel, corresponding to the number of fibres passing therethrough).

Therefore, in a first step, the images of brain magnetic resonance included in the study were segmented by means of operator-dependent procedure. This procedure was performed by using a suitably drawn toolbox K-snap—itksnap.org/pmwiki/pmwiki.php), in the three-dimensional space of resonance.

In short, the segmentation procedure consisted in identifying (and in tracking manually) glioblastoma with respect to the remaining apparently undamaged brain tissue and consequent brain oedema. To each voxel of brain a specific value was assigned; in particular, the value 0 is assigned to the voxels in which glioblastoma is absent, the value 1 was assigned to voxels corresponding to the tumour core (brain area characterized by necrosis and tumour tissue with evidence of post-contrastographic potentiation in the t1-weighed images), whereas the value 2 was assigned to voxels characterized by oedema. This procedure allowed to produce a new segmented three-dimensional image (3D) characterized by values in each part thereof.

The obtained image 3D was projected in a common space, the space called Montreal Neurological Institute (MNI), standard in the magnetic resonance analyses, through the application of different mathematic algorithms. In particular, a symmetric normalization was applied which provided a joint use of transformations of similar (stiff) and deformable (not linear) type implemented by measures of mutual information as optimization metrics (stnava.github.io/ANTs/).

As reference image or template of fibres of white matter, in the present study an atlas was created calculated upon the tractographies at 7 Tesla in MNI space of 180 subjects, belonging to a public repository called HCP (https://osf.io/5zqwg). The average template of fibres represents the average number of white matter tracts passing through each voxel of the template, still represented in 3D space and recorded in MNI space.

To the purpose of determining the new prognostic index, such reference image or template was computed through the creation, for each HCP subject, of the digital maps of tractography reconstructed by using data of diffusion magnetic resonance, data available in the above-mentioned HCP public repository. The digital maps of tractography were mediated among the included subjects to create the reference image or template of density of fibres of white matter. The voxels of such reference image or template then represent the average values of fibres passing through each one thereof, with reference to a normative sample, in this specific case represented by the data-set HCP.

With the purpose of determining the new prognostic index, as it will be illustrated in the following section, the above-described segmentation and normalization procedures can be applied even according to alternative methods. For example, the normative space can be different, as well as the template or reference image for the image calculation. The latter can be obtained by using any alternative procedure known to a person skilled in the art, for example starting from maps derived from different normative data-sets. Crucial point for determining the new index is that such reference image expresses in each space point thereof (voxel) a value corresponding to the average number of fibres of brain white matter referred to a normative sample which can derive from public data-sets and which can be generalized, or can be study-specific.

### Determination of the Prognostic Index

The method, the present invention relates to, is based upon the finding that the density of the white matter tracts is not uniform in the various brain areas and that there is a relationship between the outcome, that is the prognosis and the growth region of a brain tumour, in particular glioblastoma, depending upon the fact that this involves a brain region with high or low density of tracts.

The reference image or average template used in the study allows to quantify the number of fibres of white matter which under a normality condition pass in the growth area of glioblastoma, consisting of volume units (voxels) having each one a value corresponding to the average number of tracts crossing it. Such reference image or average template then constitutes in this context the reference standard to identify the number of fibres passing, on the average, by a specific voxel in a normative population.

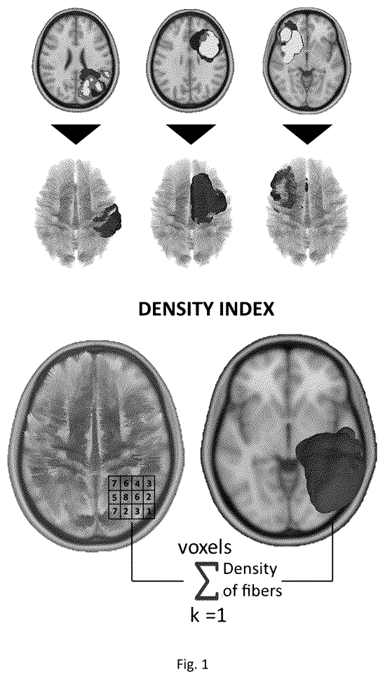

The computer implemented method illustrated hereinafter is based upon the selection, for each image of magnetic resonance of the brain of a subject affected by analysed glioblastoma, only of the voxels of the reference image or template corresponding to the brain area of the subject bearing the tumour at issue, followed by the sum of the value of all selected voxels. The result of this sum represents the new prognostic index or Tracts density Index. Such procedure is represented schematically in FIG. 1.

An example of steps of a computer implemented method for determining the new prognostic index implemented according to the present invention is provided hereinafter:
providing one or more three-dimensional images of magnetic resonance or masks of the brain of a subject affected by glioblastoma (that is three-dimensional digital objects);
providing a reference image or normative template representing the number of fibres of white matter;
binarization of the masks of tumours in which 0 corresponds to voxels free from glioblastoma and 1 to voxels included in glioblastoma (or voxels included in the tumour core);
transforming the tumour image (that is 3D object) in a unidimensional 1D vector with length equal to the number of voxels of the normalized 3D object; in other terms, each voxel of the 3D object is expressed as value of coordinate x,y,z expressing the position of the voxel in the 3D space of the object. The coordinates are mapped in a space with 1D coordinates (that is from [1,1,1] to [1]).

The maximum value [n] is represented by the maximum dimension of the number of voxels represented in the 3D space.
Applying the same space transformation to the reference image or template; the transformation does not alter the relation between the voxels in the two masks, therefore the voxels which in the 3D space of the image or mask of the tumour and of the template were represented by the same set of values [x,y,z], will assume the same value of coordinates [j] in the vector.
The values corresponding to these positions [j] are stored in a third vector.
The sum of the values of this new vector corresponds to the new prognostic index designated TDI. As mentioned in the preceding sections, the above-described method provides as initial step the binarization of the image or tumour mask (which is a three-dimensional digital object) so as to include values 0 (voxels free from glioblastoma) and 1 (voxels included in glioblastoma and, as in this case, in the adjacent oedema). The so-obtained 3D segmented image is subsequently transformed into a unidimensional vector (1D) with length equal to the number of voxels of the normalized 3D object (the same index can be calculated even for the tumour core only, as it was made in the replicative dataset, by including in value “1” only the voxels belonging to the tumour core). Then, each voxel of the 3D object is expressed as a value of coordinate x,y,z, which expresses the voxel position in the 3D space of the object. Such coordinates are mapped in a space with 1D coordinates. By taking as example a voxel in 3D space with value of coordinates [1,1,1], the transformation projects the new coordinate in the space of vector [1]. Such procedure is performed for all values of 3D space. Therefore, the minimum value of the coordinate of this space will assume value [1], whereas the maximum value [n] is represented by the maximum size of the number of voxels represented in 3D space.

Such spatial transformation is performed even for the three-dimensional object corresponding to the reference image or normative template representing the number of fibers of white matter as previously illustrated. Since such 3D object is in the same space of the 3D object of the tumour mask or image, the transformation does not alter the relationship between the voxels in the two images; therefore, the voxels which in the 3D space of the tumour image and of the reference image or template were represented by the same set of values [x,y,z] will assume the same value of coordinates [j] in the vector. Then, vectorization does not apply transformations to the value of voxels, but transforms the spatial coordinates.

This procedure allows easily to identify the position [j] in the template vector corresponding to the position [j] of the tumour image or mask which corresponds to values corresponding to 1 (indexes of a tumour tissue in 3D resonance image of brain of the considered subject). The values corresponding to these positions [j] are stored in a third vector. The sum of the values of this new vector corresponds to the new prognostic index called TDI. The vectorization and indexing procedure is illustrated in FIG. 2.

### Validity Test of the Prognostic Index Determined by the Method the Invention Relates to

The prognostic index determined in the present study with the method described in the preceding section was compared to prognostic clinical factors selected based upon literature, clinical trials and clinical practice in patients with glioblastoma. In the specific case, the variables associated to chronological age, mutation of IDH-1 gene, MGMT gene promoter methylation, performance status, treatment according to Stupp protocol (considered the standard as first line treatment, consists in the combined adjuvating treatment of radiotherapy and chemiotherapy with temozolomide) and surgery extension were included. The performance status in the discovery data-set was codified as scale score “Performance Status Eastern Cooperative Oncology Group” (PC ECOG), whereas in the replicative data-set this variable was codified through scale score “Karnofsky Perfomance status” (KPS). These scales measure the general clinical status of the patient considering the impact the disease has on his/her health and his/her level of autonomy. The surgery extension corresponds to the different levels of biopsy (total or subtotal resection). All subjects in the data-set in Berlin and everyone except one in that of Padua had a IDH wild-type genotype, therefore this variable was excluded from the analysis due to the absence of data variability. Analogously, all patients of Padua cohort were subjected to Stupp protocol, therefore this variable was inserted only in the analyses performed in the replicative data-set.

In the present study, the relation with OS (and its prediction) of the variables belonging to this group of prognostic factors was compared to the proposed prognostic index, object of patent (TDI).

The relationship between the average survival of patients affected by glioblastoma, known prognostic factors and the prognostic index TDI object of the invention, was evaluated by different statistical analyses with the purpose of analysing the prognostic utility of the new developed index. A first analysis level investigated the linear relationship between all known clinical outcomes (designated Xn) and the new TDI index. Correlation metrics of Spearman or Pearson were used based upon the distribution of the different Xn, whereas for the TDI the Pearson metric was used.

The robustness of such relationship was investigated through a procedure of leave-and-keep-one-out type. Specifically, the correlation was computed by excluding from the distribution, at each step, the piece of data of the patient with the lowest average survival value. The resulting correlations between average survival, Xn and TDI were compared with Tukey range test to evaluate which variable should allow to obtain the highest relationship with the average survival. Moreover, in order to evaluate which relationship was more stable and independent from the average survival variable (leave-and-keep-one-out), the test denominated Augmented Dickey-Fuller test was implemented.

In a second set of analyses two linear regression models were evaluated in order to evaluate the prediction level expressed in terms of explained variance; in the first model only the known prognostic factors (Xn) were included, whereas the average survival was inserted as dependent variable. In the second model even the index object of the invention (Xn+TDI) was added to the set of independent variables. In addition, such models (Xn; Xn+TDI) were studied by Cox regression to calculate the risk ratio between prognosis with Xn and TDI.

At last, the discovery data-set was divided into two different portions, corresponding to the “extreme” values of TDI (group<25° percentile; group>75° percentile) and a Kaplan-Meier analysis with log-rank test investigated differences in the survival values of these two groups.

The same analyses were performed on the replicative dataset, the only difference was that TDI was based upon the mask of the tumour core (not including the oedema, considering the availability only of the t1-weighed sequences which did not allow the segmentation of the oedematous area).

### Results

In total, in the analysis 92 patients were included in the “discovery dataset” and 58 in the “replicative dataset” aged, on the average, respectively 62±12 and 59±11 years. The average survival was 14±10 and 20±16 months in the two datasets, respectively.

A significant linear relationship with the average survival index appeared by considering ECOG performance status (r=−0.51, p<0.0001), age (r=−0.21, p=0.05) and surgery extension (r=−0.31, p=0.003) parameters, by suggesting a higher survival in young patients, with better performance status and a wider surgical resection, in line with the current literature. In addition, the results highlighted a significant relation also with TDI index, both if measured in the tumour core (r=−0.025, p=0.016), and in the tumour core and oedema (r=−0.46, p<0.001). Considering the stronger correlation when the oedema was included, the subsequent analyses were focused on TDI measured on tumour core and oedema. The relation between average survival and TDI was associated to a higher correlative score, together with the value associated to ECOG performance status. The size of glioblastoma (GBM) (both with and without oedema) did not highlight significant correlations (r=−0.011, p=0.37).

The leave-and-keep-one-out and stability analysis highlighted that only the index object of the invention showed independency from the sample and stability of results, whereas all other factors (Xn) resulted to be dependent from the average survival values of the sample.

Such results were confirmed by the (Linear and Cox) regression measurements. By adding the index (Xn+TDI) to the linear model, the explained variation increased by 10% (r2 0.40). Similar results were highlighted by Cox regression where TDI appeared as factor with the highest level of significance (p<0.005) together with the performance status. Moreover, such analysis highlighted that the risk rate is 1.09 times greater in a person with a higher TDI value than in another person.

The survival analysis according to Kaplan-Meier showed a significant difference between the two groups (log-rank p<0.005), therefore the patients with GBM at lower TDI had a longer survival. It is to be underlined that none of the patients in the group with higher values of TDI survived beyond 20 months, whereas 50% of the subjects with low values of TDI were still alive. The analysis on the replicative data-set widely confirmed the preceding results.

At last, it was tested if exemplifying cut-offs calculated on the data-set discovery and applied on the replicative data-set allowed a good OS classification of patients. To this purpose, the TDI value corresponding to 25° and 75° percentile of the TDI distribution was calculated and these values were applied to the replicative data-set, by classifying the patients as low survival or high survival (cut-off OS of 16 months). Such classification, compared with the real OS of patients of the replicative data-set, showed a (balanced) accuracy level of 87%, by confirming the prognostic utility of TDI index.

1. A computer implemented method for determining a prognostic index for a brain tumour in a subject, comprising the following steps:
a) receiving as input a three-dimensional nuclear magnetic resonance image (1) of the brain of said subject, in which said three-dimensional image (1) is segmented and recorded in a reference space and comprises a number n of voxel (10), each one having an intensity value indicative of the belonging or extraneity of the aforementioned voxel to an area of the brain bearing said brain tumour,
in which said three-dimensional image (1) is a binary image in which each voxel (10) has an intensity value equal to 1 when belonging to said area of brain affected by the brain tumour and has an intensity value equal to 0 when extraneous to the aforesaid area;
b) receiving as input a three-dimensional reference cerebral nuclear magnetic resonance image (2), in which said reference image (2) is recorded in said reference space and comprises a number m of voxels (20), in which m=n, each one bearing a value indicative of the number of tracts of nerve fibers passing through it;
in which said reference image (2) corresponds to an average of digital tractography images obtained from a plurality of healthy subjects by diffusion-weighed magnetic resonance, and in which each voxel (20) of said reference image (2) bears a value indicative of the average number of tracts of nerve fibers passing through it;
c) determining the three-dimensional coordinates of each voxel (10) of the image (1) of the brain of said subject having an intensity value indicative of the belonging of the aforementioned voxel to an area of the brain bearing said brain tumour;
d) extracting, for each voxel (20) of the reference image (2) having the three-dimensional coordinates determined in step (c), said value indicative of the number of tracts of nerve fibers passing through it;
e) adding the values extracted in step d) so as to obtain said prognostic index and comparing said prognostic index with a cut-off prognostic index determined for a group of subjects whose average survival is known,
in which, when said prognostic index has a higher value than said cut-off prognostic index, said subject is classified as having a low average survival, or, when said prognostic index has a lower value than said cut-off prognostic index, said subject is classified as having a high average survival.

2. The method according to claim 1, in which said brain tumour is an astrocytoma.

3. The method according to claim 1, in which said brain tumour is glioblastoma or glioblastoma multiforme.

4. The method according to claim 1, in which, in said three-dimensional image (1), each voxel (10) has an intensity value equal to 1 when belonging to one or more areas of the brain of said subject affected by necrosis, it has an intensity value equal to 2 when belonging to one or more areas of the brain of said subject affected by oedema, and it has an intensity value equal to 0 when extraneous to the aforesaid areas.

5. The method according to claim 1, in which said image (1) and said reference image (2) are recorded in the space of the Montreal Neurological Institute (MNI).

6. The method according to claim 1, further comprising the following steps preceding said step a):
a′) receiving as input a magnetic resonance image (1′) of the brain of said subject and subjecting it to a segmentation process so as to obtain a segmented three-dimensional image (1″) that is not normalized; and
a″) recording said segmented image (1″) that is not normalized in said reference space in such a way as to obtain said image (1).

7. The method according to claim 1, further comprising the following steps, preceding said step b):
b′) receiving as input a plurality of digital tractography images (2′) obtained from a plurality of healthy subjects and processing said images so as to obtain a reference image (2″) that is not normalized; and
b″) recording said reference image (2″) in said reference space in such a way as to obtain said reference image (2).

8. The method according to claim 1, comprising the following steps:
c′) converting said image (1) and said reference image (2) respectively into a first vector v̅ and into a second vector r̅, with length n=m, in which, for the index j which varies from 1 to n=m, each element v̅ⱼ of the vector v̅ bears an intensity value of a voxel (10) of the image (1) having a given set of three-dimensional coordinates, and each element r̅ⱼ of the vector r̅ bears a value indicative of the number of tracts of nerve fibers passing through a corresponding voxel (20) of the reference image (2) having the same set of three-dimensional coordinates of the considered voxel (10);
c″) identifying, in said vector r̅, each element r̅ⱼ that corresponds to an element v̅ⱼ having an intensity value indicative of the belonging to the voxel (10) of the image (1) to the area of the brain bearing said brain tumour;
d′) generating a third vector p with length n′ equal to the number of voxel (10) of said image (1) belonging to said area of the brain bearing said brain tumour, in which each element p̅ᵢ corresponds to said element r̅ⱼ as identified in step c″);
e′) adding the values of each element p̅ᵢ in such a way as to obtain said prognostic index.

9. The method according to claim 1, in which said tracts of nerve fibers are white matter tracts.

10. The method according to claim 1, comprising a further step of calculating the average survival of said subject on the basis of said prognostic index.

11. A non-transitory computer readable medium comprising computer-readable code on a system that when executed by a processor causes the system to implement the steps of a method according to claim 1.

12. A system, comprising:
a memory comprising computer readable instructions; and
a processor configured to read the computer readable instructions that when executed causes the system to implement the steps of a method according to claim 1.
